(19) United States
(12) Patent Application Publication
(10) Pub. No.: US 2005/0106147 A1
(43) Pub. Date: May 19, 2005

(54) Method of promoting graft survival with anti-tissue factor antibodies

(76) Inventors: Robert Jordan, Thornton, PA (US); Susan Tam, Boothwyn, PA (US); Janet Davis, Branchburg, NJ (US); Mark Zimmerman, East Brunswick, NJ (US); Gang Xu, Belle Meade, NJ (US); Norma Sue Kenyon, Miami, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/928,291

(22) Filed: Aug. 27, 2004

Related U.S. Application Data
(60) Provisional application No. 60/499,321, filed on Aug. 29, 2003.

(51) Int. Cl. A61K 39/395

(52) U.S. Cl. 424/145.1

(57) ABSTRACT

The present invention is directed to a method of using function blocking tissue factor antibodies to enhance graft survival in mammals. Function blocking antibodies having the effect of blocking activated tissue factor (TF), TF and its ligand FVII as either the inactive TF:FVII or active TF:FVIIa complex, or block the formation of the TF:FVIIa:FX ternary complex are useful in the method. These properties provide a therapy that has directed action towards thrombotic events involving tissue-plasma interactions but does not prevent the intrinsic pathway for coagulation. Activated TF arises on cells, tissues, and organs during or after transplantation and is a major cause of graft loss.

CROSS REFERENCE TO RELATED APPLICATION

This application is a non-provisional application filed under 37 CFR 1.53(b)(1), claiming priority under 35 USC 119(e) to provisional application No.60/499,321 filed Aug. 29, 2003, the contents of which are incorporated herein by reference

### BACKGROUND OF THE INVENTION

1. Field of the Invention

The present invention relates to a method of using tissue factor (TF) antagonists to promote graft survival in a patient receiving a cell, tissue or organ graft. In one aspect, the invention relates to the use of TF antagonists to promote graft survival in the preparation of pancreatic islets or use prior to implantation of pancreatic islets in a subject. The invention more specifically relates to such methods for the use of TF antagonists such as antibodies directed toward TF, including specified portions or variants thereof specific for at least one TF protein or fragment thereof, in an amount effective to inhibit the thrombotic events associated with the expression of TF on the surface of pancreatic islet preparations.

2. Tissue Factor (TF)

The coagulation of blood involves a cascading series of reactions leading to the formation of fibrin. The coagulation cascade consists of two overlapping pathways, both of which are required for hemostasis. The intrinsic pathway comprises protein factors present in circulating blood, while the extrinsic pathway requires tissue factor (TF), which is expressed on the cell surface of a variety of tissues in response to vascular injury (Davie et al., 1991, Biochemistry 30:10363). When exposed to blood, TF sets in motion a rapid cascade of activation steps that result in the formation of an insoluble fibrin clot.

TF has been investigated as a target for anticoagulant therapy. TF (also known as thromboplastin, CD142 and coagulation factor III) is a single chain, 263 amino acid membrane glycoprotein that functions as a receptor for factor VII and VIIa and thereby initiates the extrinsic pathway of the coagulation cascade in response to vascular injury. TF is an integral membrane protein normally present on the cell surface of non-vascular cell types. TF is not produced by healthy endothelial cells lining normal blood vessels, however, TF is always present in the adventitia of blood vessels.

TF serves as both a cofactor for factor VIIa, forming a proteolytically active TF:VIIa complex on cell surfaces, and as a VIIa receptor, inducing downstream intracellular changes (Bazan, J F, Proc. Natl. Acad. Sci USA (1990) 87:6934-8; Reviewed by Konigsberg, et al. Thromb. Haemost. (2001) 86:757-71). In addition to its role in the maintenance of hemostasis by initiation of blood clotting, TF has been implicated in pathogenic conditions. Specifically, the synthesis and cell surface expression of TF has been implicated in vascular disease (Wilcox et al, 1989, Proc. Natl. Acad. Sci. 86:2839) and gram-negative septic shock (Warr et al., 1990, Blood 75:1481). Furthermore, in a number of pathological states involving an acute inflammatory response and progression to a thrombotic state, such as sepsis, increased TF expression on the vascular endothelium results from the release of inflammatory mediators such as s TNF and/or IL-1.

An additional relationship between TF and inflammation has been noted in the context of glycemic control agents and insulin. WO04/041302 discloses the use of TF antagonists and blood glucose lowering agents for the treatment of thrombotic and coagulopathic diseases, respiratory and inflammatory diseases.

TF Antagonists

Various anti-TF antibodies are known. For example, Carson et al, Blood 70:490-493 (1987) discloses a monoclonal antibody prepared from hybridomas produced by immunizing mice with human TF purified by affinity chromatography on immobilized factor VII. Ruf et al, (1991, Thrombosis and Haemostasis 66:529) characterized the anticoagulant potential of murine monoclonal antibodies against human TF. The inhibition of TF function by most of the monoclonal antibodies that were assessed was dependent upon blocking the formation or causing the dissociation of the TF/VIIa complex that is rapidly formed when TF contacts plasma. Such antibodies were thus relatively slow inhibitors of TF in plasma as factor VII/VIIa remains active. One monoclonal antibody, TF8-5G9, was capable of inhibiting the TF/VIIa complex by blocking the F.X binding site without dissociating the complex, thus providing an immediate anticoagulant effect in plasma which is not absolute as F.VII is still available. This antibody is disclosed in U.S. Pat. Nos. 6,001,978, 5,223,427, and 5,110,730. Ruf et al. suggest that mechanisms that inactivate the TF/VIIa complex, rather than prevent its formation, may provide strategies for interruption of coagulation in vivo. In contrast to other antibodies that inhibit factor VII binding to TF, TF8-5G9 shows only subtle and indirect effects on factor VII or factor VIIa binding to the receptor. TF8-5G9 binds to defined residues of the extracellular domain of TF that are also involved in F.X binding with a nanomolar binding constant. Thus, TF8-5G9 is able to effectively block the subsequent critical step in the coagulation cascade, the formation of the TF:VIIa:X ternary initiation complex (Huang et al, J. Mol. Biol. 275:873-894 1998).

Anti-TF monoclonal antibodies have been shown to inhibit TF activity in various species (Morrissey et al, Throm. Res. 52:247-260 1988) and neutralizing anti-TF antibodies have been shown to prevent death in a baboon model of sepsis (Taylor et al, Circ. Shock, 33:127 (1991)), and attenuate endotoxin induced DIC in rabbits (Warr et al, Blood 75:1481 (1990))

WO 96/40921 discloses CDR-grafted anti-TF antibodies derived from the TF8-5G9 antibody. The TF8-5G9 antibody humanized by CDR-grafting disclosed in WO96/40921 was subsequently designated CNTO 859 by the applicants. Other humanized or human anti-TF antibodies are disclosed in Presta et al, Thromb Haemost 85:379-389 (2001), EP1069185, U.S. Pat. No. 6,555,319, WO 01/70984, WO03/029295, and WO04/039842.

The Role of TF in Transplantation

Despite the central role of TF in blood coagulation, the mechanisms underlying the regulation of TF pro-coagulant activity in vivo are still being explored as are non-coagulant activities related to receptor signaling (Morrissey, J. H. Thromb Haemost 2001; 86:66-74 and Key, N. S., Bach, R. R. Thromb Haemost 2001; 85:375-6).

Unperturbed cells in culture have weak coagulant activity, however, cells or tissues that have been disrupted or stimulated with e.g. growth factors or endotoxin leading to increased intracellular calcium ion (Ca++) display fully expressed and active TF. Perturbation of the phospholipid species between the inner and outer cell membrane leaflets, especially phosphatidyl serine, was implicated as a possible trigger of this de-encryption of a macromolecular substrate binding site on TF which defines TF activation (Bach, R. R, Moldow, C. F. Blood 1997; 89 (9): 3270-3276).

A number of reports implicate the role of active TF in the pathogenesis of transplant failure. U.S. Pat. No. 6,387,366 notes that bone marrow stem cell (BMSC) transplantation causes blood clotting and/or hemorrhage due to the expression of TF on the infused cells and suggests several methods to reduce the biological activity of TF or FVII in infusions employing BMSC transplantation, gene therapies employing BMSC, and other types of cell transplantation. These methods include treating the preparation or the patient with TF antagonists.

Veno-occlusive disease (VOD) is the most common regimen-related toxicity accompanying stem cell transplantation (SCT). Severe VOD complicated by multisystem organ failure (MOF) remains almost uniformly fatal. Defibrotide, a single-stranded polydeoxyribonucleotide that has specific aptameric binding sites on vascular endothelium, has shown promise in the treatment of VOD. Among other actions, Defibrotide modulates tissue factor expression by microvascular endothelial cells. (Falanga A, et al. Blood 1999; 94:146a).

Cyclosporine, a common agent used to prevent graft rejection in patients receiving all types of organ transplants, has been shown to inhibit tissue factor expression in monocytes and macrophages (Hoelshermann, H. et al. Blood 1996; 10: 3837-3845).

Islet cell transplant has come to be used to treat severe cases of pancreatitis and for diabetes. Transplantation of islets rather than the whole pancreas is attractive because of its technical ease and the ability to transplant early in the course of the disease. Islets are more than single cells and less than a complete organ in that they are comprised of several cell types in somewhat organized structures. However, the transplant of autologous or allogenic islet tissue alike is often unsuccessful due to the thrombotic events taking place upon introduction of the graft material to the patient (via the portal vein). It has been noted that tissue factor is expressed on cells in these preparations and that anti-tissue factor antibodies can reduce the coagulation time of these preparations when introduced into plasma (Moberg et al. Lancet 2002, 360: 2039-2045).

Cardiac allograft vasculopathy (CAV) is the cause of death for 20% of heart transplant patients who have survived more than 3 to 5 years and is associated with early deposition of fibrin. A study undertaken to determine the role of TF in CAV demonstrated increased TF staining on the luminal surface to beyond the medial layers of small intramyocardial arteries in biopsy samples taken from transplanted hearts and TF score was associated with a greatly increased incidence of CAV. This research suggested but did not use TF antagonists including anti-TF antibodies as potential treatments for the prevention or amelioration of CAV in heart transplant patients (Yen, M. H. et al., Circulation 2002, 106: 1379-1383).

Thus, there is a clear medical need for peri-transplant treatments that diminish or eliminate the risk of thrombosis. There is also a need for methods to treat or ameliorate the effects seen in grafts surviving longer term but at risk due to increased TF activation. The therapy must be both efficacious; preventing thrombosis at the graft-blood interface, and safe; having minimal risk of hemorrhagic events.

### SUMMARY OF THE INVENTION

The present invention relates to a method of using antagonists of human tissue factor such as anti-TF antibodies, for enhancing graft survival and function in patients receiving transplanted organs, cells, or tissues. In one embodiment, the tissue factor antagonists are used in accordance with the invention for the enhancement of graft survival and function in islet cell transplantation. The method of using the antagonists includes treating the donor organ; the prepared transplantation cells, tissue, or whole organ,prior to contact with the patient; or treating the patient with the antibody prior to contact with the transplantation material.

In a preferred embodiment, the invention relates to use of antibodies to tissue factor, particularly those which are function blocking antibodies capable of preventing the proteolytic cleavage of FX to FXa by FVIIa, to enhance graft survival and function in organ or tissue transplantation particularly islet cell transplantation. Such antibodies are particularly suited for this use, being highly competent function blocking antibodies in so far as they prevent the downstream events of the extrinsic pathway of blood coagulation, while not preventing the normal functions of the intrinsic factor pathways. The antibodies thereby afford an enhanced measure of safety from hemorrhagic complications when treating a human patient. Thus, in this embodiment, the invention provides a method of enhancing graft survival and function in patients receiving transplanted cells, particularly islet cells, organs or tissues, which comprises (a) treating the donor cells, organs or tissue with an antibody to tissue factor, prior to transplantation of such cells, organ or tissue, and/or (b) treating the patient prior to transplantation of such cells, organ or tissue with an antibody to tissue factor in an amount effective to enhance graft survival and function. Optionally, the treatment method may also comprise continued treatment of the patient with anti TF antibody after transplantation.

The invention further discloses a method for preventing graft rejection in patients treated with inductive or maintenance doses of immunosuppressive agents in connection with receipt of grafted cells, tissue, organs, digits or limbs. The method combines the use of function blocking anti-tissue factor antibodies in combination with the use of immunosuppressive agents administered to a patient, most preferably, non-steroidal immunosuppressive agents, to prevent graft rejection.

### BRIEF DESCRIPTION OF THE DRAWINGS

FIG. 1 shows photomicrographs of isolated human islet preparation stained with CNTO859 (left and center panel) or non-specific human IgG (left panel) followed by FITC-labeled goat anti-human antibody and viewed using fluorescence optics.

FIG. 2A is a graph showing the relationship between islet number in 200 microL volume and coagulation time when citrated plasma was added to the islets. FIG. 2B shows the relative increase in coagulation time affected by additon of 10 Ug/ml CNTO 859 to about 7500 islets in 200 microL for 1 h prior to addition of citrate plasma.

FIG. 3 is a series of three overlaid tracings of the optical density over time (recorded in seconds) for samples of Cynomolgus islet-culture media prepared three different ways and incubated with or without 50 microgm/microL CNTO 859 prior to addition of citrated plasma. FIG. 3A is media that was subjected to centrifugation at 1200 rpm for 5 min. FIG. 3B is media that was centrifuged at 14,000 rpm for 10 min. FIG. 3C is media that was filtered using a 0.2 micron pore size filter.

FIG. 4 is a bar graph comparing the prothrombin time (PT) measured in a representative pair of 10 monkeys where the solid bars represent data from the systemically treated monkey.

FIG. 5A-D are graphs showing fasting C-peptide and rapamycin levels in transplanted monkeys over time.

FIG. 6 is a bar graph showing the calculated T½ to reach maximum coagulation density for various cell lineages under the same assay conditions with or without the indicated concentration of CNTO 859 in micrograms per ml.

### DETAILED DESCRIPTION OF THE INVENTION

A number of pathologies involving the complications arising from the activation of TF on the surface of transplanted cells, tissues, or in the vasculature of transplanted organs are improved by treatment with TF antagonists in the method of the present invention. While the events leading to activation of TF in the cellular components of living materials prepared for human patients are largely unavoidable, blocking the functions of TF in inducing coagulation are possible using the antibodies of the present invention. Thus while events such as mechanical trauma, exposure to unnatural surfaces on the vessels and implements used and the release of cellular contents including Ca2+, which lead to the activation of TF cannot be avoided in transplantation, the resulting coagulation can be blocked by the method of invention. In addition, the activation of stress pathways (causing for example, release of nitric oxide and secretion of inflammatory cytokines) can induce immediate up-regulation of TF expression, either during organ/cell harvest from donor/culture or shortly after implantation/infusion into recipient. The effects of the increased TF expression can be addressed by the present invention.

Transplanted Cell, Tissue, and Organs

The method is applicable to the transplantation of a variety of living materials. These living materials include cells, such as stem cells of various lineages; tissue, such as pancreatic islet preparations; or organs, such as heart or kidneys. Generally, any type of autologous or allogeneic tranplant wherein the transplanted materials will be in direct contact with the circulation is amenable to the methods of the present invention.

Organs; skin, kidney, liver, heart, pancreas, bone marrow, small intestine, and lung can be taken from brain-dead donors and prepared for transplantation. In some cases, partial hepatectomy tissue can be transplanted from a living donor to a recipient. Digits or limbs may be autografted or re-attached. In recent medical history, an entire forearm has been transplanted with success. While technical feasibility of allografting or xenografting organs or limbs has been overcome, the risk of rejection has been the major consideration in the success of these procedures. As new immunosuppressive agents and regimens are proved, grafting has and will continue to expand. The appropriate preparation of the patient and graft to avoid unwanted coagulation provides further assurance against ischaemic complications.

As stated, the method is particularly applicable to pancreatic islet cell transplantation. For allografts of islet cells, islets can be prepared from pancreatic tissue taken from brain-dead multiorgan donors. The pancreatic duct of in situ washed organs is cannulated and liberase enzyme (Boehringer Mannhaim, Indianapolis, Ind.) is perfused. The enzymatically digested pancreas is further mechanically dissociated as needed and the islets separated on a refrigerated Cobe-2991 centrifuge (Cobe BCT, Lakewood, Colo.). The pooled islets can be either stored refrigerated or kept in culture before being infused into the patient.

In a recent description, stem cells can be used to prepare insulin responsive tissue or “psuedo”—islet like aggregates by induction and differentiation of stem cells in culture (U. Florida WO 0326584). Alternatively, the appropriate stem cells can be infused directly into the patient.

TF Antagonists

As used herein, the term “TF antagonists” refers to a substance which inhibits or neutralizes the activity of TF or F.VIIa or the active TF:VIIa complex. Such antagonists accomplish this effect in a variety of ways. One class of TF antagonists will bind to TF protein with sufficient affinity and specificity to neutralize the function of TF. Included in this class of molecules are antibodies and antibody fragments (such as for example, F(ab) or F(ab′)₂ molecules). Thus, as used herein “antibody” refers to immunoglobulin molecules and immunologically active portions of immunoglobulin molecules. Another class of TF antagonists are fragments of TF protein, muteins or small organic molecules i.e. peptidomimetics, that will bind to TF, thereby inhibiting the activity of TF. The TF antagonist may be of any of these classes as long as it is a substance that inhibits TF pro-coagulation activity in so far as the formation of the TF:FVIIa:FX ternary complex is prevented. TF antagonists include certain TF antibodies, modified TF, antisense TF and partial peptides of TF, and F.VIIa inhibitors.

Anti-TF Antibodies

Only the anti-TF antibodies known in the art that meet the criteria of function blocking antibodies may be employed in the method of the present invention. Included are the murine monocolonal antibodies to TF in U.S. Pat. Nos. 6,001,978; 5,223,427; and 5,110,730. WO 96/40921, discloses CDR-grafted anti-TF antibodies derived from the TF8-5G9 antibody in which the complementary determining regions (CDR's) from the variable region of the mouse antibody TF8-5G9 are transplanted into the variable region of a human antibody and joined to the constant region of a human antibody. The antibody described therein is referred to as CNTO859 throughout this application. Other humanized anti-TF antibodies with similar characteristics are disclosed in Presta et al, Thromb Haemost 85:379-389 (2001) and EP1069185 and U.S. Pat. No. 6,555,319. Each of the foregoing references are incorporated by reference into the present application.

Compositions and Their Uses

The function blocking anti-TF monoclonal antibody can be used to prevent the anti-coagulant action of TF associated with the transplanted material or with the host response to the transplanted material in accordance with the invention. The individual to be treated may be any mammal and is preferably a human patient in need of such treatment. The amount of monoclonal antibody administered will vary according to the type of transplant it is being used for and the method of administration.

The TF antibodies of the invention of the present invention may be administered by any number of methods that result in the enhanced survival of graft material in the host. Further, the anti TF antibodies of the invention need not be present locally to impart an effect, therefore, they may be administered wherever access to body compartments or fluids containing TF is achieved. The source material, the source donor, or the prepared material to be transplanted may be treated by direct application of a formulation containing the antibodies. In the alternative, the patient or recipient of the transplant may be administered the antibodies of the invention prior to, during or after the transplant is made. The latter methods include intravenous administration of a liquid composition, transdermal administration of a liquid or solid formulation, oral, topical, interstitial or inter-operative administration.

In a preferred embodiment, the anti-TF antibody is administered systemically by pre-treatment of the patient undergoing pancreatic islet transplantation and optionally, combined with treatment at various intervals post-transplantation. In-this method, the antibody is preferably administered within I hour, preferably 10-20 minutes, prior to intrahepatic islet infusion, optionally combined with continuous treatment for up to 14 days post transplantation or longer to prevent graft rejection. Administration may also be oral or by local injection into a vein or artery supplying blood to the intended site for deposition of the transplanted cells, tissues or organ such as, to the portal vein in the case of pancreatic islet cell transplantation. However, generally, the monoclonal antibody is effectively administered intravenously. Generally, the dosage range is from about 0.05 mg/kg to about 12.0 mg/kg. This may be as a bolus or as a slow or continuous infusion which may be controlled by a microprocessor controlled and programmable pump device.

For the ex vivo treatment of donor material (cells, organs or tissue), the method comprises incubation of the antagonist or antibody with the donor material prior to transplantation. In the case of anti-TF antibody, donor material is incubated with anti-TF antibody at a concentration of about 0.1-100 μg/ml, preferably about 10-50 μg/ml for a period of about 0.5-72 hours.

Alternatively, DNA encoding preferably a fragment of said monoclonal antibody may be isolated from hybridoma cells and administered to a mammal. The DNA may be administered in naked form or inserted into a recombinant vector, e.g., vaccinia virus in a manner which results in expression of the DNA in the cells of the patient and delivery of the antibody.

The monoclonal antibody used in the method of the present invention may be formulated by any of the established methods of formulating pharmaceutical compositions, e.g. as described in Remington's Pharmaceutical Sciences, 1985. For ease of administration, the monoclonal antibody will typically be combined with a pharmaceutically acceptable carrier. Such carriers include water, physiological saline, or oils.

Formulations suitable for parenteral administration include aqueous and non-aqueous sterile injection solutions which may contain anti-oxidants, buffers, bacteriostats and solutes which render the formulation isotonic with the blood of the intended recipient; also aqueous and non-aqueous sterile suspensions which may include suspending agents and thickening agents. Except insofar as any conventional medium is incompatible with the active ingredient and its intended use, its use in any compositions is contemplated.

The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use.

Combinations with TF Antagonists

The method may be carried out by combining the TF antagonists of the invention with one or more other agents having the effect of enhancing graft survival or reducing immune rejection of the transplant.

The most commonly used agents given to patients before and after transplantation procedures act to suppress the body's ability to mount an immune response to an antigens on the transplanted cells or tissue that might be recognized as foreign. Glucocorticosteroids such as prednisolone or cortisone are very often a component of the pre- and post-transplant regimen for immunosuppression. Glucocorticoids, while capable of modifying the body's immune responses to diverse stimuli, also have the drawback of causing profound and varied metabolic effects. In particular, they act as insulin antagonists, raising blood sugar.

The fungal derived product, cyclosporine, is also widely used agent in pre- and post-transplant or graft management. Cyclosporine sold under the names SANDIMMUNE (Novartis), NEORAL (Novartis) and GENGRAF (Abbott) exerts multiple effects on the immune system and is a potent immunosuppressive agent that in animals prolongs survival of allogeneic transplants. Cyclosporine has been demonstrated to suppress some humoral immunity and to a greater extent, cell-mediated immune reactions such as allograft rejection, delayed hypersensitivity, experimental allergic encephalomyelitis, Freund's adjuvant arthritis, and graft vs. host disease in many animal species for a variety of organs. Cyclosporine specifically and reversibly arrests the cell cycle of immunocompetent lymphocytes. T-lymphocytes are preferentially inhibited. The T-helper cell is the main target, although the T-regulatory cells may also be suppressed. Cyclosporine also inhibits lymphokine production and release including interleukin-2.

Another approach to preventing acute graft rejection involves the administration of an antibody recognizing CD3 on T-cell surfaces. One such product made by Ortho Biotech, Raritan, N.J. is ORTHOCLONE OKT3 (muromomab-CD3).

Combining anti-inflammatory or anti-cytotoxic therapy with anti-tissue factor agents in the peri-transplant setting is expected to be particularly effective due to the aforementioned association of elevated TF in patients with diabetes. Such agents include glucocorticoids or COX-2 inhibitors, with the caveat that the hyperglycemic actions of corticosteroids must be taken into account in the treatment of diabetic patient.

For islet cell transplantation, a non-diabetogenic regimen excluding glucocorticoids has proven effective. This regimen, referred to herein as the “Edmonton protocol” (Shapiro, A. M. et al. N. Engl. J. Med. 2000; 343: 230-238, 2000) includes sirolimus (RAPAMUNE, Wyeth-Ayest), tacrolimus (FK506, PROGRAF, Fujisawa), and an anti-IL2R Mab daclizumab (ZENAPAX, Roche). These agents work to suppress T-lymphocyte proliferation and activity by a variety of mechanisms. Tacrolimus inhibits T-lymphocyte activation. Sirolimus inhibits T lymphocyte activation and proliferation that occurs in response to antigenic and cytokine IL-2, IL-4, and IL-15 stimulation by a mechanism that is distinct from that of other immunosuppressants. Sirolimus also inhibits antibody production. In cells, sirolimus binds to the immunophilin, FK Binding Protein-12 (FKBP-12), to generate an immunosuppressive complex. The sirolimus:FKBP-12 complex has no effect on calcineurin activity. This complex binds to and inhibits the activation of a key regulatory kinase. This inhibition suppresses cytokine-driven T-cell proliferation, inhibiting the progression from the G1 to the S phase of the cell cycle. Daclizumab, by blocking the T-cell IL-2 receptor of active but not resting T-cell, also prevents T-cell responses to antigenic challenge.

As mentioned above, the Edmonton protocol for islet transplantation has proved beneficial in that it avoids steroids as immunosuppressive agents in favor of the more specifically T-cell directed agents including; sirolimus, tacrolimus, and daclizumab.

Abbreviations

FVII—Factor VII (proenzyme)

FVIIa—Factor VIIa (active)

FX—Factor X (proenzyme)

FXa—Factor Xa (active)

hIg—Human Ig

While having described the invention in general terms, the embodiments of the invention will be further disclosed in the following examples.

### EXAMPLE 1

### Presence of Tissue Factor on Isolated Islets but not Normal Pancreatic Tissue

A normal human pancreas was obtained and cryosectioned by a trained pathologist. Sections were stained with a murine anti-human tissue factor antibody, C632, and for insulin, Ab-6 (NeoMarkers, Inc.) as a positive control and with anti-human IgG (Jackson Labs) as a negative control. The binding was detected using a kit (DAKO Biotin-Link, Streptaviden-HRP). Sections were counter-stained with hematoxylin (H).

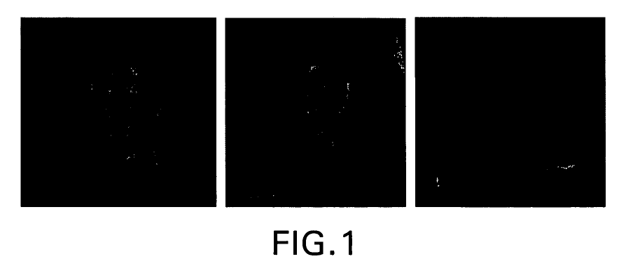

In the H stained sections, the islets were clearly identified at 100× magnification (FIG. 1 left panel). Insulin staining was positive, however, no staining with anti-TF was observed. In contrast, TF staining was present in the epithelial cells of the pancreatic duct in the same section.

To prepare isolated islets, the donor organ is harvested and islets obtained via the semi-automated method of Ricordi (Ricordi C., et al. Diabetes 1988 37: 413-420, 1988). Briefly, islets are released by means of protease perfusion and purified using Ficoll gradient centrifugation. Islets can be maintained in culture medium. The human islet preparation in this example contained 70% viable cells at the time of the study.

Human islets were incubated with 20 ug/ml CNTO 859 for 1 H at RT, washed once, and incubated with FITC conjugated anti-human IgG (Jackson Labs), washed twice and mounted on a microscope slide using a CytoSpin instrument. Samples were viewed using a Nikon fluoresecent microscope with a phase contrast objective. The islets stained with CNTO 859 were uniformly fluorescent over the entire surface (FIG. 1 center panel). Those stained with anti-hIgG were dimly stained (FIG. 1 right panel).

Owing to the difficulty in obtaining human pancreatic islets for experimental use, experiments using Cynomolgus monkey islets were performed. CNTO 859 has been previously shown to be able to bind cyno-TF. Cynomolgus islets were incubated with 20 ug/ml CNTO 859 for 1 H at RT washed once in HBSS and stained in the same manner as the human islets. TF was detected on the surface of the islets under 400× magnification.

### EXAMPLE 2

### Anti-Coagulant Effects of CNTO 859 on Islets

The donor organ is harvested and islets obtained via the semi-automated method of Ricordi (Ricordi C., et al. Diabetes 1988 37: 413-420, 1988). Briefly, following in situ vascular flushing with cold solution, human cadaveric pancreata were removed from brain-dead multiorgan donors, islets are released by means of perfusion through the pancreatic duct with Liberase enzyme (Boehringer Mannheim, Indianapolis, Ind.) and purified using Ficoll gradient centrifugation. Islets can be maintained in culture medium for some hours until transplantation. Both human and cynomolgus monkey islets were harvested and transferred from laboratory to laboratory by commercial carrier.

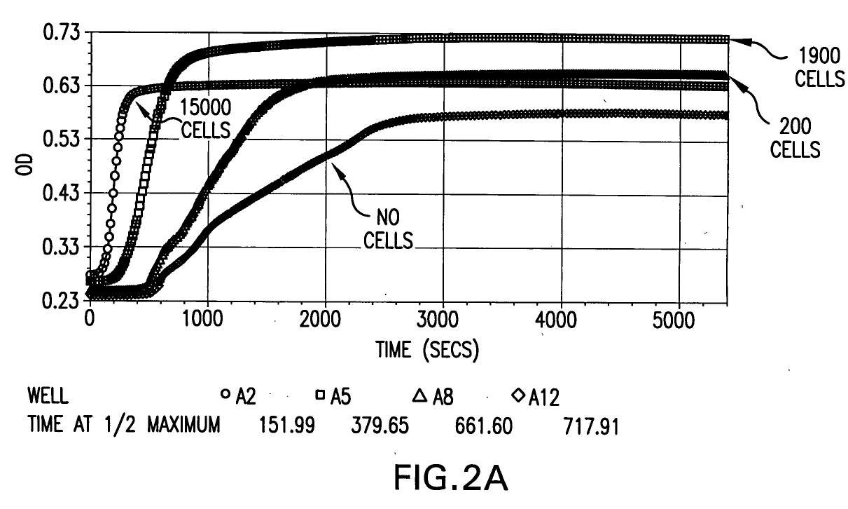

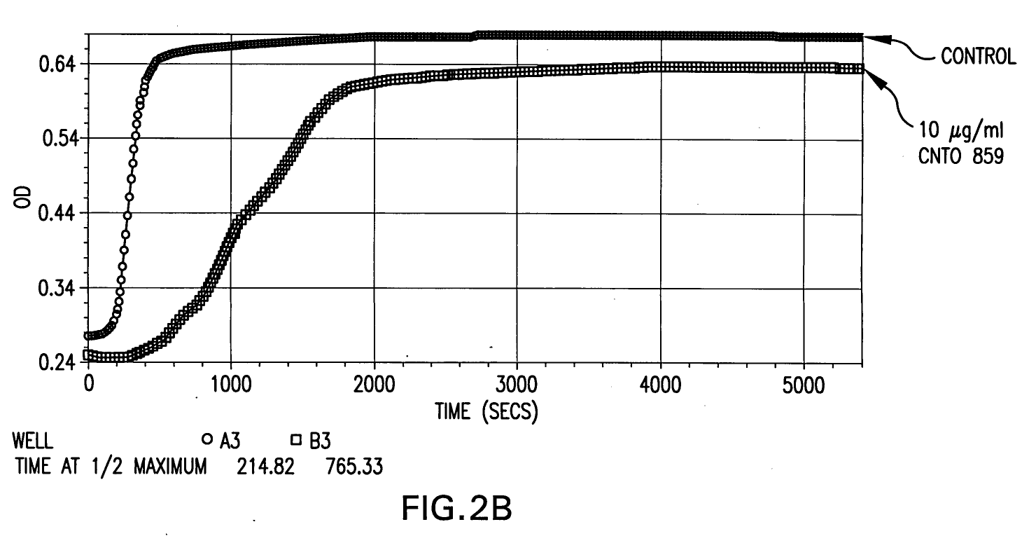

Plasma Clotting Assay Human islets were serially diluted from 30,000 cells/well of a 96-well plate. CaCl₂ (11 mM) was added to citrated plasma and 60 ul of the plasma was added to sample wells. Clotting was monitored for 1.5 H at 37° C. in a recording ELISA reader at 405 nm. At this wavelength, the turibidity of the sample is a measure of clotting. Clotting times (time to maximal OD change) was cell number dependent: faster with more cells per well and prolonged with fewer cells as compared to no cells. The times to ½ maximum OD were calculated from the curve of OD v time as: 152 s for 15,000 cells, 380 s for 1900 cells, 662 s for 200 cells, and 718 s for no cells (FIG. 2A).

Human islets (7500 in 200 ul of HBSS without Ca2+ or Mg2+) were pre-incubated with 10 ug/ml CNTO 859 for 1 H at RT in a 96-well plate. Citrated plasma was added (as above) and the clotting reaction monitored. The time to ½ maximun was calculated as: 215 s without antibody and 765 s with CNTO 859 (FIG. 2B).

Cynomolgus monkey islets were determined 95% viable at the time of preparation.

This example demonstrates the ability of anti-tissue factor IgG antibody to prolong coagulation time when islets are introduced into plasma.

### EXAMPLE 3

### Release of TF from Islets Preparations in Culture

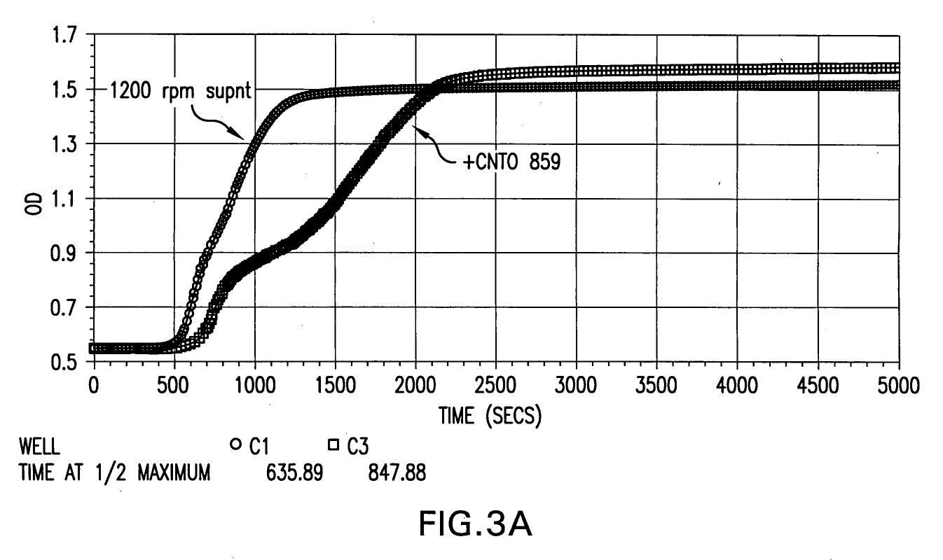

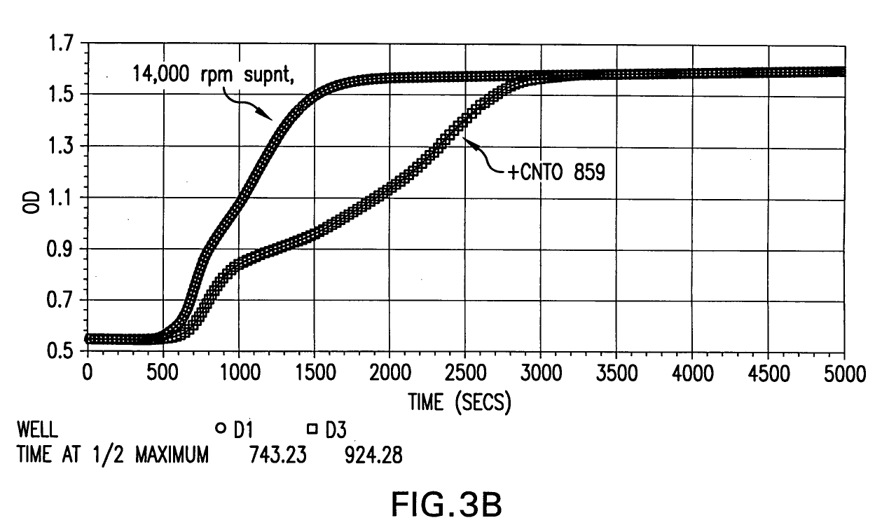

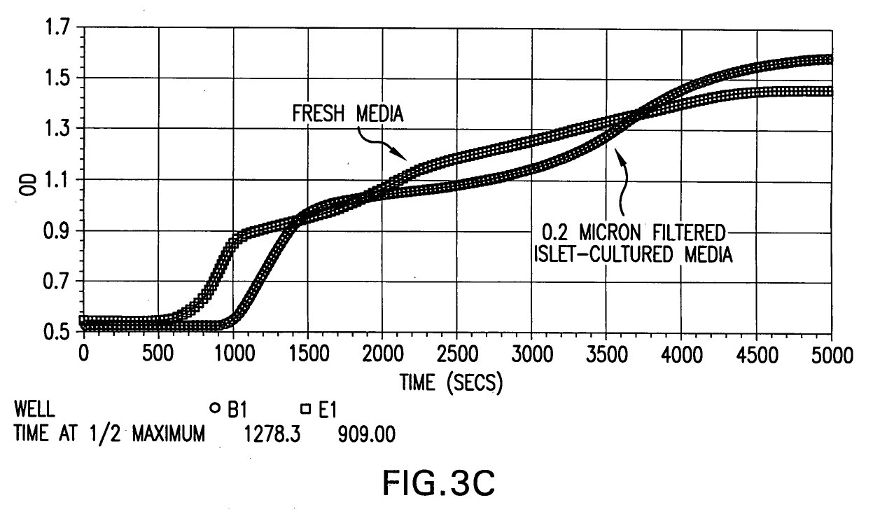

Cynomoglus islet-cultured media (from shipment) was either 0.2 micron filtered, centrifuged at 1200 rpm, for 5 minutes or 14,000 rpm, for 10 minutes. Samples were incubated with buffer or CNTO 859 (50 ug/ml) for 1 h and then tested in the plasma clotting assay. Only the filtered medium did not stimulate coagulation. Reduced time aggregation (reduced clotting time) was seen in the presence of the islet-cultured media that had been centrifuged at low or high speed which was reversed by the addition of CNTO 859 (FIG. 3A & B). Media that had been filtered did not accelerate plasma clotting time and CNTO859 had no effect upon it.

No detectable soluble TF was observed using the TF commercial ELISA kit (Immubind Tissue Factor, American Diagnostica).

These results indicate that TF is released in microparticles from islets held in culture medium. Shed membrane apoptotic microparticles rich in PS, an activator of TF, are produced in considerable amounts within human atherosclerotic plaques and may determine plaque thrombogenicity (Mallet et al. Circulation (1999) 99:348-353). Cell-derived microparticles with procoagulant potential were detectable days following in the circulating blood of patients with recent clinical signs of plaque disruption and thrombosis. These microparticles are implicated in the ongoing prothombotic state of those patients (Mallet et al. Circulation (2000) 101: 841). However, TF containing microparticles have not heretofore been reported derived from pancreatic islets.

### EXAMPLE 4

### Prolonged Protection by CNTO 859 Treated Islets Against Plasma Coagulation

A quantitative measure of the effectiveness of CNTO 859 (50 ug/ml, 1 h, and washed) in preventing thrombosis was performed using treated cynomolgus islets or human J82 cells. Islets or cells were incubated with 50 ug/ml CNTO 859 for 1 h, washed, incubated with fresh media for various times at 37° C., and tested in the plasma clotting assay. Each sample was tested in duplicate. On day 4, CNTO 859 appeared to be more effective in prolonging plasma clotting (TABLE 1).

Islets (˜1250) were incubated with ¹²⁵-I CNTO 859 (5 uCi/ug, 50 ug/ml) in NHP islet media for 2 h at RT, then washed 3× with HBSS. Washing was done by microfuging islets at 1000 rpm for 2 min, then aspirating the supernatant. Islets were resupended in 500 ul NHP islet media, and at various time points, duplicate 50 ul supernatant samples were removed for counting in the gamma counter. On day 4, islets were pelleted, supernatant removed, and the bottom of the microfuge tube was cut and counted for islet-bound cpm. The pellet cpm and supernatant cpm at day 4=total cpm in the reaction mixture. For calculations, supernatant cpm/total cpm×100%=% loss of bound ¹²⁵-I CNTO 859 from islets.

TABLE 1

| | Relative % Increase in | | |
|---|---|---|---|
| | Coagulation Time | | % ¹²⁵I-CNTO 859 Lost from |
| Day | J82 Cells | Cynomolgus islets | Cynomolgus Islets |
| 0 | 100 | 100 | 0 |
| 1 | 33 | 37 | 59 |
| 2 | 20 | 23 | 66 |
| 4 | 100 | 56 | 99 |

125-I CNTO 859 was used to show that islets, once treated and washed, slowly release antibody. As the incubation conditions were the same as for the functional assay, the data indicate that CNTO 859 can functionally inhibit islet initiation of plasma clotting for more than 24 hours when the treated islets are introduced into fresh plasma. On day 4, CNTO 859 treated islets showed better inhibition of plasma clotting. CNTO 859 “pacified” islets (by downregulating signaling proteins, inflammatory molecules, etc) so that islets became less thrombotic. Viability of islets on day 4 was estimated to be >70% in a parallel experiment. The time course of the effect was similar on the J82 cells.

### EXAMPLE 5

### In Vivo Islet Transplantation Protocol Using CNTO 859

Studies were designed to test the effect of humanized anti-tissue factor on allogeneic islet engraftment and long-term survival in a cynomolgus monkey marginal mass model. The goal of the experiments iwas to define a protocol that results in insulin independence with reduced numbers of transplanted islets based on the Edmonton protocol and to demonstrate enhanced graft survival and lack of hemorrhagic events using subject pre-treatment with CNTO 859.

Efficacy is gauged by decreased coagulation parameters as well as the reduction in inflammatory markers such as cytokine production, in treated vs. untreated monkeys

The following tests are performed on plasma collected from citrate anticoagulated blood:
Prothrombin time (verifies effect of circulating CNTO 859)
APTT (to test for heparin anticoagulation)
Thrombin-antithrombin (TAT) complex ELISA
Fibrin D-dimer ELISA
Prothrombin fragment 1+2 (F1.2) ELISA
IL-6 ELISA
IL-10 ELISA
CRP ELISA

Samples for these assays are collected pre-diabetes induction, pre-therapy SFIS (steroid free immune suppression) and pre-treatment with anti-TF/pre-transplant (antibody is given as a single bolus 10-20 minutes prior to intrahepatic islet infusion) and at 1 hr, 4 hrs, 8-12 hrs, 24 hrs, 48 hrs, and 7 and 14 days post-transplant.

The monkeys must undergo one month of quarantine, then one month to induce and verify diabetes, and 6 months of post-transplant follow-up.

Diabetes induction and management in the cynomolgus monkey: Recipients are 1-3 year old cynomolgus monkeys of Mauritian origin; donors of the same strain will be >4 years of age. Prior to diabetes induction, all recipients undergo IVGTT to obtain baseline data; diabetes is induced with 1250 mg/m² streptozotocin. Diabetic animals are treated with NPH insulin before the morning meal and with NPH/Lantus before the afternoon meal to maintain blood glucose levels between 100-200 mg/dl. Four weeks after diabetes induction, the monkeys undergo a glucagon challenge to verify that no c-peptide is being produced.

Experimental Groups: There is 1 arm of the study, with 3 pairs of recipient monkeys, all treated with SFIS. Each recipient of a pair receive half of the islet mass from the same single donor. One animal in each pair is treated in vivo with TF antibody and is infused with TF antibody pre-treated cultured islets; the other recipient is treated with SFIS (steroid free immune suppression) alone and receives islets cultured with or without TF antibody. Islet isolation and transplantation: The donor organ is harvested and islets obtained via the semi-automated method of Ricordi (Ricordi C., et al. Diabetes 1988 37: 413-420, 1988). The recipient monkeys then undergo a mini-laparotomy and the islet transplantation is performed via the portal vein with infusion into the liver.

A pair of monkeys receive approximately 5,000 IEQ/kg from the same donor; one monkey receives the experimental intervention with CNTO 859 and the other does not. For the antibody treated monkey, the antibody is infused at a dose of 6 mg/kg 10-20 minutes prior to islet infusion. For both monkeys, rejection is prevented with steroid free immune suppression (low dose FK506, high dose rapamycin, anti-IL2R induction therapy) beginning the day prior to transplant, as is clinically employed and has previously demonstrated efficacy in cynomolgus monkeys.

Animals are monitored twice a day to determine fasting and post-prandial blood glucose levels (heel stick, glucometer) and treated with insulin as needed to maintain blood glucose in the 100-200 mg/dl range. The dose of insulin required is recorded. Fasting c-peptide is determined every other week and intravenous glucose tolerance testing (IVGTT) is undertaken every 8 weeks to determine FPIR.

Efficiency of engraftment: Enhanced engraftment in monkeys treated with α-TF experimental interventions and steroid free immune suppression (SFIS) versus those that receive SFIS alone can be verified by:
1) maintenance of normoglycemia in the presence of continued minimal insulin doses, and possibly, attainment of insulin independence with a marginal mass;
2) maintenance of c-peptide production over several months of follow-up; and
3) enhanced c-peptide responses to intravenous glucose tolerance testing over several months of follow-up.

The data is considered in the context of maintenance of therapeutic levels of rapamycin (15-20 ng/ml trough levels in this model). Based on previous experience in monkey models, monkeys were followed for up to 6 months or more, as it can take several months to observe graft loss that is caused by attrition of islets due to a marginal mass (the model used here). This is most likely due to the need for revascularization, which may mask differences in islet quality over the short term.

Results

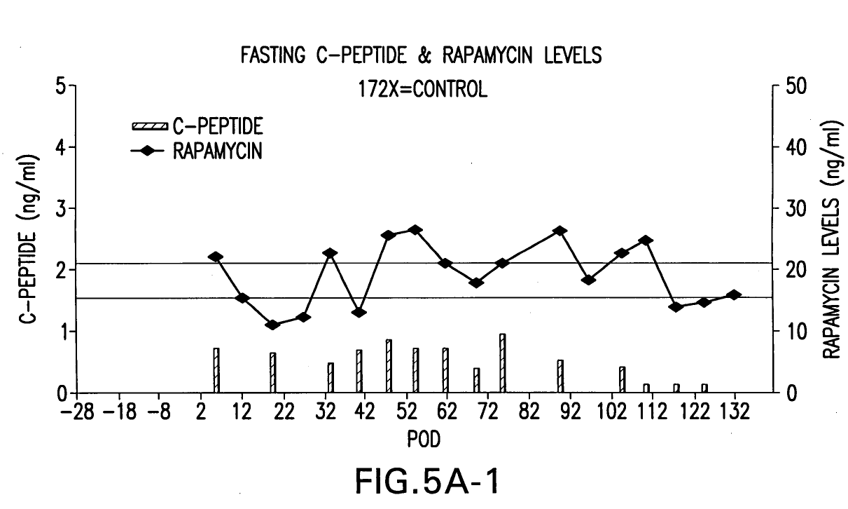

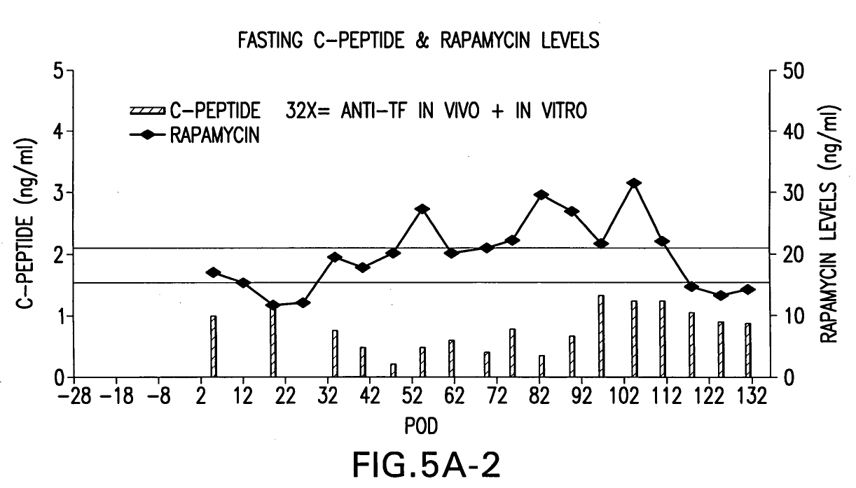

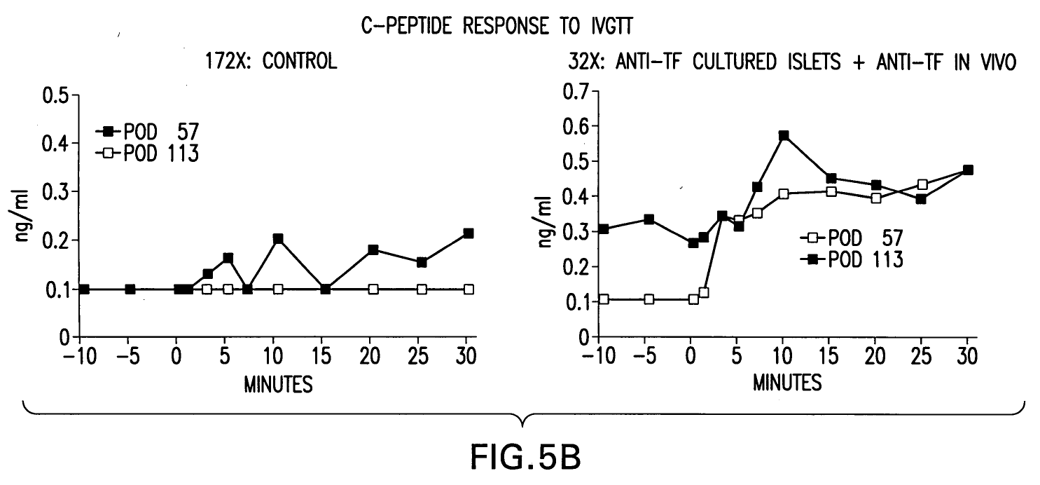

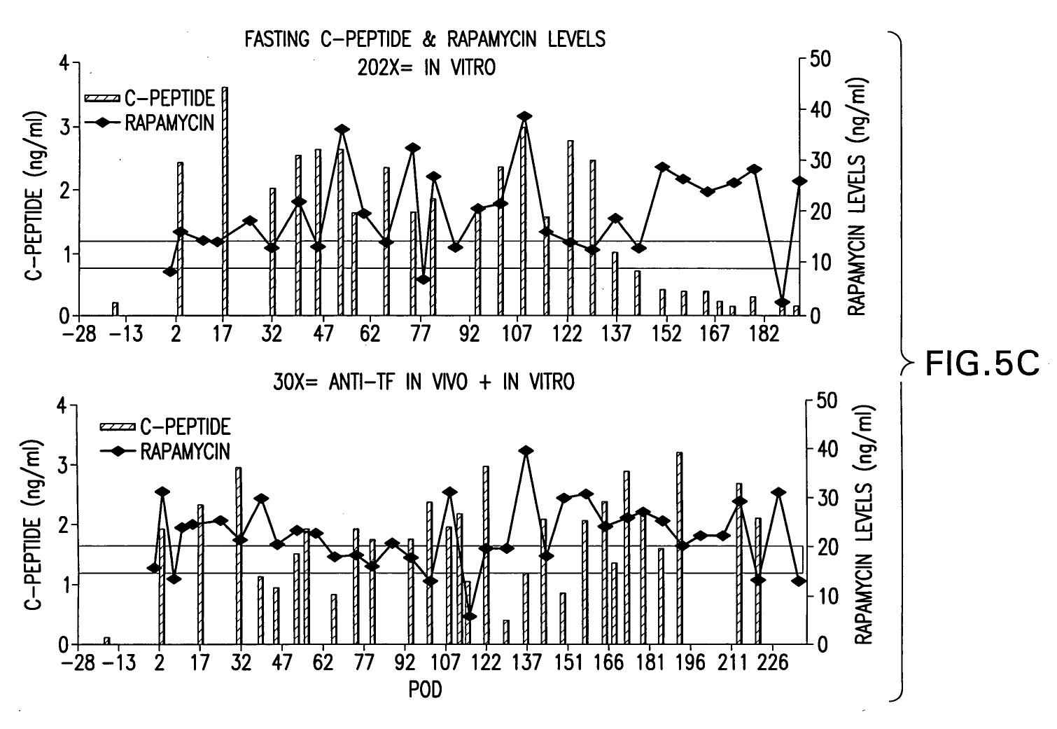

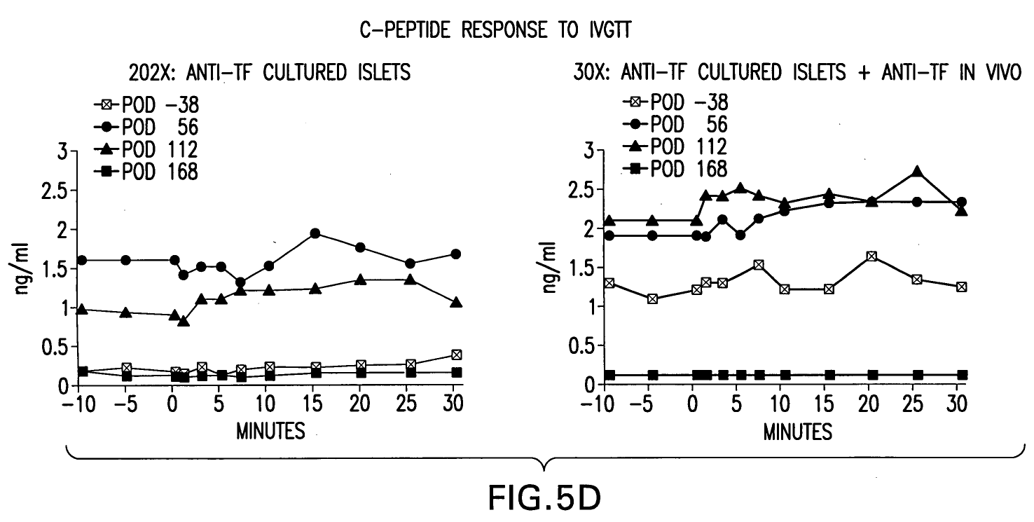

Three pairs of monkeys were transplanted. For one pair, both animals lost graft function at approximately 3 months, despite adequate rapamycin levels (data not shown), thereby suggesting that the initial islet prep was of poor quality in this experiment. This also points out the importance of simultaneously transplanting islets from the same donor into both control and treated animals, thereby eliminating the possibility that differences among treated and nontreated monkeys are due to variations in islet quality. The results for the other two monkey pairs were highly informative. While trends towards enhanced glycemic control in the presence of reduced levels of exogenous insulin doses were observed for in vivo anti-TF treated monkeys, the clearest demonstration of the efficacy of in vivo anti-TF for enhancement of islet engraftment can be seen by comparing the fasting c-peptide levels in transplanted monkeys over time (FIGS. 5a and 5b).

As shown in FIG. 5A, both the control monkey (172X) and the in vivo anti-TF treated monkey that received islets cultured in anti-TF (32X) experienced islet function in the first 3 months post-transplant, as determined by fasting c-peptide levels. Both had adequate levels of rapamycin. Despite the fact that both animals received islets from the same donor islet preparation, the control monkey lost function (c-peptide decreasing to <0.1 ng/ml) at approximately post-operative day (POD) 100, while the treated animal has maintained excellent islet function (c-peptide production) beyond POD 130. In FIG. 5B, it can be seen that 32X has good c-peptide production in response to IVGTT at POD 57 and increased production on POD 113. In striking contrast, monkey 172X has abnormal c-peptide responsiveness to IVGTT, and is negative by POD 113.

As shown in FIG. 5C, similar results were obtained for a pair of monkeys in which the control monkey was not treated in vivo with anti-TF but received islets treated with anti-TF (202X), while the experimental animal received both in vivo anti-TF and in vitro anti-TF treated islets (30X). Both monkeys demonstrated good fasting c-peptide production in the first 4 months post-transplant; however, monkey 202X gradually lost function by approximately 6 months post-transplant, while the in vivo treated monkey 30X has maintained excellent function greater than 7 months post-transplant. As shown in FIG. 5D, monkey 202X produced c-peptide in response to IVGTT early on but was negative on POD 168, while monkey 30X has continued to produce good c-peptide levels (The apparent lower c-peptide levels on POD 168 are not considered significant, as the starting level that day was lower and can be influenced by glucose starting value).

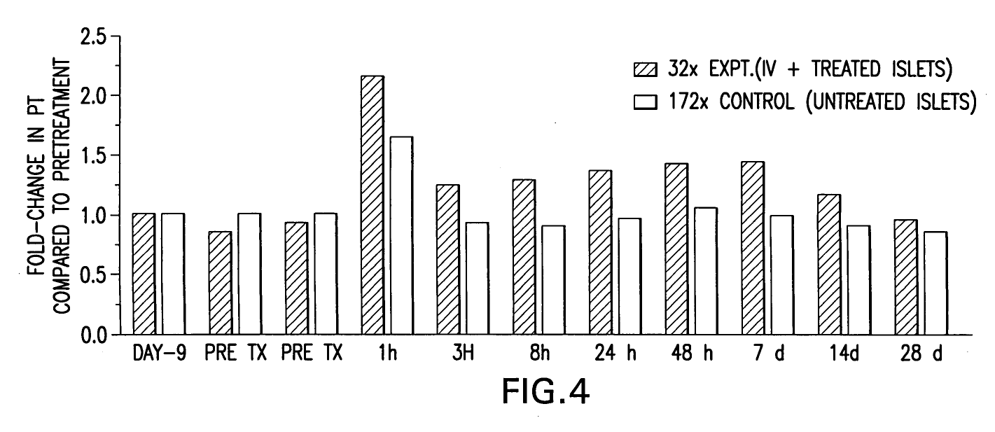

The prolongation of coagulation time afforded by the dose of CNTO 859 used was verified using a two-stage PT assay; TF (cynomolgus brain TF) is incubated with plasma sample prior to adding CaCl₂ to initiate the clotting reaction. The difference in prothrombin time (PT) in one of the monkeys given systemic CNTO 859 is shown in FIG. 4. These data also demonstrate the longevity of the effect of a single administration of CNTO 859 in prolonging PT.

This data shows a marked difference in islet survival and function for in vivo anti-TF treated recipients. It also demonstrates the critical importance of using pairs of monkeys that receive islets from the same donor. It may be possible that in vitro anti-TF alone allows for some improvement in engraftment (compare length of function for animal pairs in 5A and 5C); however, it may also be possible that islet quality for the monkeys in 5C was superior to that for 5A recipients.

Islets pre-treated in culture ex vivo with CNTO 859 for time periods ranging 24-48 hours showed no loss in viability or glucose responsive function, indicating no detrimental toxicities from antibody exposure. The prolongation of coagulation time seen with plasma samples from treated monkeys can be viewed as proof of principle that circulating levels of antibody can be achieved in vivo, sufficient to inhibit coagulation of TF expressing cells, without associated adverse events. Early islet loss during the immediate post-transplant phase may encompass multiple complications involving inflammation, apoptosis, and complement-mediated cytotoxicity in addition to coagulation. Therefore, combinations of agents, not single intervention strategies, may be necessary for improved transplant outcome.

### EXAMPLE 6

### Human Ductal Pancreatic Cells

In human islet preparations, ductal epithelial cells are present in a greater proportion than in islets prepared in the same manner from the pancreas of cynomologus monkeys. As was discovered in EXAMPLE 1, human pancreatic ductal cells stain positively for TF in situ prior to disruption of the tissue. Ductal epithelia of other organs are presumed to similarly display TF. Therefore, we wished to titrate the expression of TF in human ductal cells.

An established human pancreatic ductal cell line (Capan-1, ATCC) was used for the assay. A human bladder cancer cell line, J82 (ATCC) was used as a positive control. The latter cell line has been calculated to display approximately 500,000 CNTO 859 binding sites per cell.

The plasma clotting assay was performed essentially as described in Example 2. Cells were lightly trypsinized from culture on the day of assay, washed and counted before adjusting a final cell suspension to 10⁶ cells/ml in HBSS. The assay was performed in a flat-bottom 96-well plate, using 10⁵ cells per well. Antibody (CNTO 859) or control diluent was added at 5× concentration in a volume of 25 ul/well and allowed to pre-bind to the cells for 30 minutes at room temperature. CaCl₂ was added to each well (25 ul of 150 mM stock solution) along with 100 ul of citrated, pooled human plasma to initiate the reaction. Coagulation was monitored at room temperature in a kinetic plate reader using 15 sec interval readings at 405 nm over 60-120 minutes. A final reading of T_{1/2} maximal clotting time was obtained for each sample. Controls for the assay included Simplastin (liquid thromboplastin) and HBSS buffer without cells. The assay was performed with replicates of eight yielding CVs of <10%.

The results are expressed in terms of time to reach half maximal OD in secs (Table 2 ). CNTO 859 at as low as 1 ug/ml prolonged coagulation time of human plasma inititated by ductal pancreatic cells.

TABLE 2

| Sample | Simplastin | Capan-1 Cells | Cells + 1 ug/ml | Cells + 5 ug/ml | Cells + 10 ug/ml | Cells + 20 ug/ml | HBSS Control |
|---|---|---|---|---|---|---|---|
| ½ Max | 72 | 429 | 661 | 723 | 746 | 765 | 2458 |
| (Secs) | | | | | | | |

### EXAMPLE 7

### Investigation of TF Density on Stem Cell Tissue Sources

Mesenchymal stem cells (MSC) were isolated from whole bone marrow purchased from Cambrex BioScience Walkersville, Inc (www.cambrex.com). Cells were obtained from 10-ml aspirates taken from the iliac crest of normal human donors (age about 20 years). Mononuclear cells were isolated by gradient centrifugation. All cells were plated at a constant density of 10,000 cells/cm2 on 15-cm culture dishes in DMEM-low glucose medium containing 5% fetal bovine serum and 100 U/ml penicillin and 1000 U/ml streptomycin. After seven days, nonadherent cells were discarded. Colony-forming units were isolated by cloning rings and plated at low densities for further expansion.

Umbilical and Placental Sources. Following normal childbirth, postpartum tissue was obtained from National Disease Research Interchange, Philadelphia, Pa. The umbilicus or placenta tissues were washed extensively in phosphate buffered saline to remove blood. The tissue was minced using scalpels and digested with collagenase (Clostridium histolyticum; Sigma, St Louis, Mo.), dispase (bacillus polymyxa; Invitrogen Grand Island, N.Y.) and hyaluronidase (bovine testis; Sigma). Cells dissociated from the tissue were centrifuged and extensively washed in growth medium composed of low-glucose Dulbecco's modified Eagle's medium (Invitrogen), 15% v/v fetal bovine serum (Hyclone, Logan, Utah), 0.001% v/v betamercaptoethanol (Sigma), 50 units per milliliter penicillin and 50 micrograms per milliliter streptomycin sulfate (Invitrogen). Viable nucleated cells were seeded in growth medium at 5,000 cells per square centimeter on flasks previously coated with a 2% w/v porcine gelatin solution (Sigma). When cells reached confluence they were passaged using 0.05% w/v trypsin-EDTA (Invitrogen) every 3-4 days. Approximately two population doublings were achieved per passage.

Equivalent numbers of cells in a 96 well plate were compared in a plasma clotting assay. Cells were resuspended in HBSS buffer (100 ul) and CaCl₂ adjusted to 15 mM. Citrated normal pooled plasma (100 ul) was added to start the clotting reaction. Coagulation was monitored in a kinetic plate reader at 405 nm, RT, with 15 sec reads for 1 h. Samples were tested in triplicates. Time to half maximal clotting (T ½), was obtained for each sample.

For the initial evaluation of TF density on the cells, samples contained ˜2500 cells/well. Generally, on viewing results from all cell dilutions, there was a 2-fold delay in (T½) clotting with placental cells compared to J82 (tumor) cells. Another way to think about results is: it would take ˜50× more placental cells to have the same clotting potential as a highly thrombotic (J82) tumor cell. Generally, at all the different cell dilutions, there were similar clotting kinetics for umbiblical cells and J82 cells.

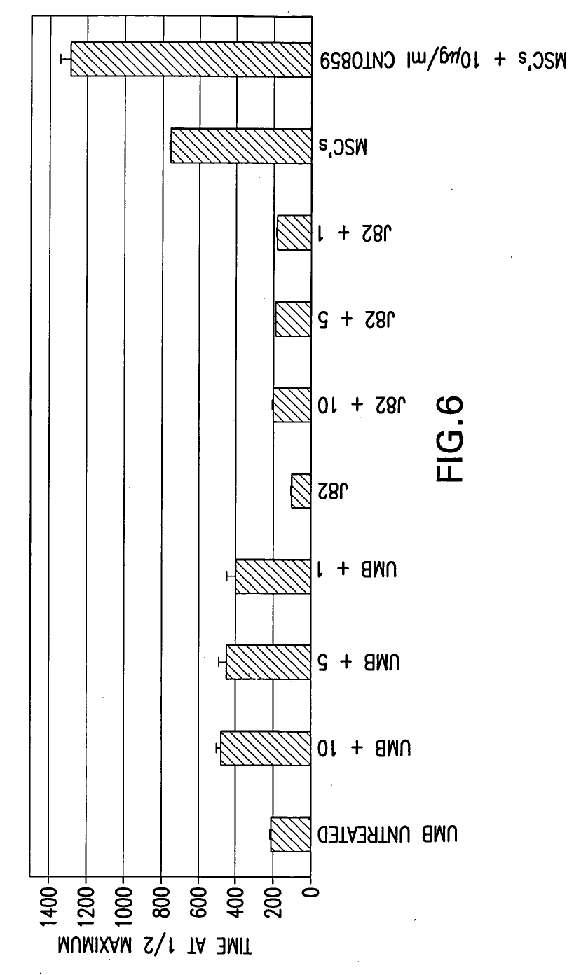

The ability of CNTO 859 to prolong coagulation time as initiated by the presence of cells of various lineages was also tested. FIG. 6 shows the calculated T½ for umbilical cells (UMB), bladder cancer cells (J82), and mesenchymal stem cells (MSC) in the presence and absence of various concentrations of CNTO 859. While the cancer cell line has the highest pro-coagulant activity, UMB cells also have significant potential, more than mesenchymal stem cells.

1. A method for enhancing the survival of a graft in a patient receiving said graft comprising contacting said graft with an antibody that binds to tissue factor and prevents The formation of the TF:FVIIa:FX ternary complex

2. The method of claim 1 wherein the antibody competes with the antibody CNTO 859 for binding to tissue factor.

3. The method according to claim 1, in which the antibody is a Fab, Fab′, or F(ab′)2 fragment or derivative thereof.

4. The method of claim 1 where the graft is an organ, a stem cell, or a tissue preparation.

5. The method of claim 4 where the graft is a pancreatic islet cell preparation.

6. The method of claim 4 where the antibody is present during the isolation of the organ, stem cell or tissue preparation.

7. The method of claim 4 where the antibody is added to the organ, stem cell or tissue preparation prior to transplant of said preparation to said patient.

8. The method of claim 4 where the antibody is administered to the patient prior to the transplantation of graft within one hour prior to transplantation.

9. The method according to claim 2, in which the monoclonal antibody is administered intravenously to the patient.

10. The method according to claim 9, in which the monoclonal antibody is administered in the amount of from 0.05 mg/kg to 12.0 mg/kg body weight.

11. The method according to claim 10, in which the monoclonal antibody is administered in a bolus dose followed by an infusion of said antibody.

12. The method of any of claims 1-11 wherein the antibody is administered in combination with an immunosuppressive agent.

13. A method of claim 12 where the immunosuppressive agent agent is selected from the group consisting of cyclosporine, tacrolimus, sirilimus, and daclizumab.

14. The method of claim 12, where the immunosuppressive agent blocks the CD3 receptor on T-cells.

15. A method for enhancing the survival of a pancreatic islet cell graft in a patient which comprises: (1) pre-treating the patient with an anti-tissue factor antibody and (b) continuing the treatment post-transplantation for a period of up to 14 days, or longer to prevent graft rejection.

16. A method for enhancing the survival of a stem cell graft in a patient which comprises: (1) pre-treating the patient with an anti-tissue factor antibody and (b) continuing the treatment post-transplantation for a period of up to 14 days, or longer to prevent graft rejection.

17. The method of any of claims 1-11 wherein the antibody is administered in combination with an anti-inflammatory or anti-cytotoxic agent.

18. The method of claim 17 wherein the anti-inflammatory agent is a glucocorticoid or COX-2 inhibitor.

19. The method of claim 4 where the graft contains human ductal pancreatic cells.

20. The method of claim 5 where the pancreatic islet preparation contains human ductal pancreatic cells.
